(19) United States
(12) Patent Application Publication
(10) Pub. No.: US 2007/0118009 A1
(43) Pub. Date: May 24, 2007

(54) Method for controlling the application of ionizing radiation to a body

(75) Inventor: Martin Hoheisel, Erlangen (DE)

Correspondence Address: SIEMENS CORPORATION;INTELLECTUAL PROPERTY DEPARTMENT, 170 WOOD AVENUE SOUTH, ISELIN, NJ, 08830, US

(73) Assignee: SIEMENS AKTIENGESELLCHAFT

(21) Appl. No.: 11/474,580

(22) Filed: Jun. 26, 2006

(30) Foreign Application Priority Data
Jun. 27, 2005 (DE) 10 2005 029 893.1

(51) Int. Cl. A61N 5/00

(52) U.S. Cl. 600/3

(57) ABSTRACT

To implement a method for controlling the application of ionizing radiation to a body, provision is made for an instrument, which can apply therapeutic radiation sources to a body, with a part of a locating system being provided to locate the position of the therapeutic radiation sources on an instrument in the body. With the instrument inserted into the body of the patient, an assignment of the located position of the therapeutic radiation sources to the three-dimensional representation of the body is possible by using the locating system. The therapeutic radiation source can then be applied, when the located position corresponds to a position previously determined on the basis of the three-dimensional representation of the body.

### CROSS REFERENCE TO RELATED APPLICATIONS

This application claims priority of German application No. 10 2005 029 893.1 filed Jun. 27, 2005, which is incorporated by reference herein in its entirety.

### FIELD OF THE INVENTION

The invention relates to a method for controlling the application of ionizing radiation to a body. It thus pertains to the field of brachytherapy. With brachytherapy, radioactive emitters are incorporated into the body to be treated so as to deliver a localized high radiation dosage and thus destroy malignant tissue. This can be effected by implanting seeds, i.e. radioactive emitter which remain in the body. Alternatively, a so-called afterloading can be carried out, in other words, a radiation source can be inserted and removed again. In both cases, it is necessary to position the radiation source as near as possible to the tumor.

### BACKGROUND OF THE INVENTION

The contour of the tumor is usually determined on the basis of a three-dimensional image, for instance a computed tomography image or an image generated by means of magnetic resonance. The emitter and/or applicator (instrument with the emitter) is inserted using fluoroscopic control or with the aid of ultrasound imaging. In the prior art it is difficult to position the radiation source accurately relative to the position of a tumor previously located on the basis of the three-dimensional representation.

### SUMMARY OF THE INVENTION

The object is thus to provide a method for controlling the application of ionizing radiation to a body, which, compared with the prior art, particularly allows better location of a therapeutic radiation source.

To achieve the object, the invention provides a method and an instrument according to the claims.

The method according to the invention thus comprises the steps:
creating a three-dimensional representation of the body,
providing an instrument, which can apply therapeutic radiation sources to a body, with a part of a locating system for locating the position of the therapeutic radiation sources on the instrument in the body,
inserting the instrument with therapeutic radiation sources into the body while simultaneously locating the position of the therapeutic radiation sources with the (complete) locating system,
assigning the located position of the therapeutic radiation sources to the three-dimensional representation of the body,
applying the therapeutic radiation source, when the located position corresponds to a position previously determined in the three-dimensional representation of the body.

The invention is thus essentially based on the use of a locating system which is linked to the applicator instrument. The three-dimensional position of the applicator can thus be determined during the insertion of the applicator into the body, and the treating doctor is no longer restricted to a control by means of ultrasound or fluoroscopic imaging. Ultrasound and fluoroscopic imaging can however also be used in addition.

The three dimensional representation is preferably created with the aid of computed tomography, C-arm computed tomography, magnetic resonance tomography or three-dimensional sonography.

The instrument can be constructed like conventional applicators in the prior art of brachytherapy and must only have space for the part of the locating system. Reference is made here to a “part of a locating system”, because the locating system comprises a part of the instrument and a part located outside the body of the patient, with the signals being emitted in at least one direction. For example, an electromagnetic locating system can feature a device outside the patient body, which serves as a source for electromagnetic radiation and an evaluation unit which detects the electromagnetic radiation received by a coil on the instrument (further details are not discussed here in terms of the lines connecting the coil to the outside world). The instrument used to apply therapeutic radiation sources to a body can comprise a rigid needle, in which the therapeutic radiation source is located, with this needle then being connected to the part of the locating system. It can also feature a flexible tube, in which the therapeutic radiation source is located, with this flexible tube then being connected to the part of the locating system.

To relate the signals received by the locating system to the position of the instrument, it may be necessary for the locating system to know the position of the body in the patient space. To this end, marker elements can be fixed to the body, which are to be identified in the three-dimensional representation of the body. If the locating system is then designed to determine the position of the marker objects, the mapped marker elements can be made to correspond to the located marker elements on the images, so that it is possible to assign the located position of the instrument to the three-dimensional representation of the body.

The assignment of the located position of the therapeutic radiation sources to the three-dimensional representation of the body can also include the therapeutic radiation sources or even the part of the instrument with which they are applied (instrument tip) being displayed in the three dimensional representation. A corresponding arrow can be marked on the three-dimensional image for instance, so that the treating doctor can detect where the therapeutic radiation sources are located at present on the basis of the three-dimensional representation serving to determine the tumor contours.

The instrument according to the invention is an instrument which can be inserted into the body of a patient in order to transport therapeutic radiation sources, as used conventionally with brachytherapy, and comprises in accordance with the invention a part of a locating system such that the position of the tip of the instrument can be determined with the complete locating system. It is thus implicitly assumed here that the therapeutic radiation sources are applied with the tip of the instrument, i.e. that it is important to determine the position of the tip, so as to establish where the therapeutic radiation sources reach, if they are emitted by the instrument into the body of the patient with the instrument in its current position.

The instrument can be a catheter, the tip of which is connected to an electromagnetic receiving coil, which is part of an electromagnetic locating system. It can also be a rigid instrument, whereby with such rigid instruments a part of the rigid instrument conventionally protrudes out of the body of the patient. Optical markers are then attached to this part as part of an optical locating system. The complete locating system then comprises an emitter, which emits light and a receiver, which receives a signal from the optical markers.

### BRIEF DESCRIPTION OF THE DRAWINGS

A preferred embodiment of the invention is now described with reference to the drawing, in which;

FIG. 1 shows a patient examination with the instrument according to the invention.

### DETAILED DESCRIPTION OF THE INVENTION

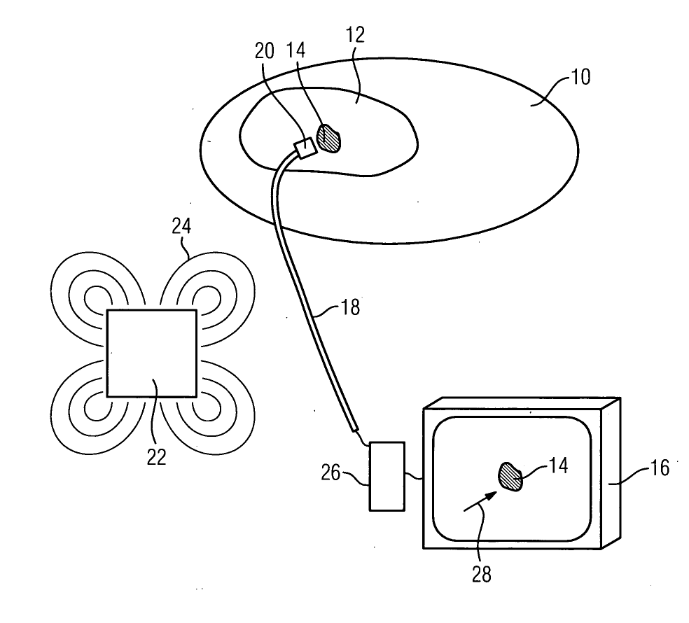

FIG. 1 shows a schematic cross-sectional representation of patient 10. The patient 10 has an organ 12 in which a tumor 14 is located. The tumor 14 was previously identified and pinpointed with the aid of a three-dimensional representation of the body of the patient 10. The image data from the three-dimensional representation is stored on a computer system (not shown) and the tumor 14 is displayed on a screen 16.

The tumor 14 is now treated brachytherapeutically, i.e. the tumor tissue is to be destroyed by means of radioactive radiation. To this end a radioactive emitter, i.e. a therapeutic radiation source, must be brought close to the tumor 14. A catheter 18 is used for this purpose. The catheter 18 takes the form of a typical instrument (applicator) used for applying therapeutic radiation sources, as known from the prior art. In addition, in accordance with the invention, an electromagnetic receiving coil 20 is attached to the catheter 18, said receiving coil being part of a locating system. A further part of the locating system is a generator 22 of electromagnetic radiation 24 only shown schematically in FIG. 1. The electromagnetic radiation 24 then induces a voltage in the receiving coil 20, said voltage being supplied to an evaluation unit 26. If the form of the electromagnetic radiation is suitable, it is known from the prior art that the three-dimensional position of the coil 20 can be located. If the position of the patient table is known, on the basis of the marker elements for instance, the tip of the catheter 18 can be schematically displayed in the three-dimensional representation on the screen 16 by an arrow 28. The treating doctor thus tracks the position of the tip of the catheter 18 on the basis of the arrow 28 on the screen 16, so as to obtain information about how close the catheter 18 is to the tumor 14. If the catheter 18 is arranged sufficiently close to the tumor 14, the therapeutic radiation source is emitted from the catheter 18 to the body 10.

As already mentioned, the catheter 18 is a conventional application catheter for therapeutic radiation sources. The electromagnetic locating system comprising the generator 22 and the receiving coil 20 with the evaluation unit 26 is known as such in the prior art (c.f. for instance the article by P. G. Seiler et al., “A novel tracking technique for the continuous precise measurement of tumor positions in conformal radiotherapy”, Phys. Med. Biol., Volume 45 (2000), pages N103-N110). The invention firstly provides the link between such a locating system and an application catheter 18, whereby it is possible to locate the tip of the catheter 18 and assign it to the three-dimensional representation displayed on the screen 16. This enhances the usefulness of the three-dimensional representation. It is not only used beforehand to diagnose the tumors, but also during the operation. Fluoroscopic images or ultrasound images generated during the operation as well can also be displayed on the screen 16, with the necessity for generating these images essentially diminishing due to the good support for the treating doctor on the basis of the representation of the arrow 28 on the screen 16.

1-10. (canceled)

11. A method for controlling an application of radiation to a human medical patient body, comprising:
creating a three-dimensional representation of the body;
providing an instrument with a therapeutic radiation source which applies the radiation to the body;
inserting the instrument into the body;
arranging a locating system connectable to the instrument for locating a position of the therapeutic radiation source;
assigning the located position of the therapeutic radiation source to the three-dimensional representation of the body; and
applying radiation to a treating area of the body when the located position of the therapeutic radiation source corresponding to a position of the treating area previously determined on the three-dimensional representation of the body.

12. The method as claimed in claim 11, wherein the three-dimensional representation is created by the method selected from the group consisting of: computed tomography, C-arm computed tomography, magnetic resonance tomography, and three-dimensional sonography.

13. The method as claimed in claim 11,
wherein the instrument comprises a rigid needle with which the therapeutic radiation source is located,
wherein the rigid needle is connected to a part of the locating system, and
wherein the part of the locating system is an electromagnetic receiving coil.

14. The method as claimed in claim 11,
wherein the instrument comprises a flexible tube with which the therapeutic radiation source is located,
wherein the flexible tube is connected to a part of the locating system, and
wherein the part of the locating system is an electromagnetic receiving coil.

15. The method as claimed in claim 11, wherein the locating system comprises an electromagnetic radiation source, an electromagnetic receiving coil, and an electromagnetic radiation evaluation unit.

16. The method as claimed in claim 11, wherein an optical marker is attached to the body for locating a position of the body and is identified in the three-dimensional representation of the body.

17. The method as claimed in claim 11, wherein the position of the therapeutic radiation is displayed on the three-dimensional representation of the body.

18. The method as claimed in claim 11, wherein a part of the instrument connected with a part of the locating system is displayed on the three-dimensional representation of the body.

19. The method as claimed in claim 11, wherein the radiation is an ionizing radiation.

20. The method as claimed in claim 11, wherein the treating area is a tumor.

21. An instrument for applying a therapeutic radiation to a treating area of a human medical patient body, comprising:
a therapeutic radiation source attached to the instrument inserting into the patient body for emitting the therapeutic radiation to the treating area of the patient body; and
a locating system connected to the instrument for locating a position of the therapeutic radiation source.

22. The instrument as claimed in claim 21, wherein the instrument is a catheter and a part of the locating system which is an electromagnetic receiving coil is connected at the tip of the catheter.

23. The instrument as claimed in claim 22, wherein the instrument is rigid and a part of the locating system which is an electromagnetic receiving coil is connected at the tip of the rigid instrument.

24. The instrument as claimed in claim 23, wherein the locating system comprises an electromagnetic radiation source, an electromagnetic receiving coil, and an electromagnetic radiation evaluation unit.

25. The instrument as claimed in claim 24, wherein an optical marker is attached on the instrument for identifying a position of the patient body.

26. The instrument as claimed in claim 21, wherein the treating area is a tumor.
